(19) United States
(12) Patent Application Publication
(10) Pub. No.: US 2024/0374123 A1
(43) Pub. Date: Nov. 14, 2024

(54) MEDICAL IMAGING DEVICE, IN PARTICULAR A STEREO ENDOSCOPE OR STEREO EXOSCOPE

(71) Applicant: KARL STORZ SE & CO. KG, Tuttlingen (DE)

(72) Inventors: Lukas BUSCHLE, Mühlheim (DE); Werner Göbel, Überlingen (DE)

(73) Assignee: KARL STORZ SE & CO. KG, Tuttlingen (DE)

(21) Appl. No.: 18/557,284

(22) PCT Filed: Apr. 25, 2022
(86) PCT No.: PCT/EP2022/060863

§ 371 Date: Oct. 25, 2023

(30) Foreign Application Priority Data
Apr. 26, 2021 (DE) 10 2021 110 611.7

(51) Int. Cl. A61B 1/00; A61B 1/06

(52) U.S. Cl. CPC A61B 1/00186; A61B 1/000094; A61B 1/00193; A61B 1/0638

(57) ABSTRACT

A medical imaging device, in particular a stereo endoscope or stereo exoscope, includes a first light source and a second light source; a first optical path having a first image sensor having a first sensor filter; and a second optical path having a second image sensor having a second sensor filter; wherein the first optical path and the second optical path are spatially offset from one another, and a first image and a second image form a superposed image having a dual piece of image information. The particular light source is designed to illuminate the viewing region with a particular light spectrum such that physiological parameters of the viewing region can be determined on the basis of the particular light spectrum and different spectral ranges of the particular image can be evaluated to obtain an additional piece(s) of image information regarding the physiological parameters in the viewing region.

### CROSS-REFERENCE TO RELATED APPLICATIONS

This application is the U.S. national stage of PCT/EP2022/060863 filed on Apr. 25, 2022, which claims priority of German Patent Application No. 10 2021 110 611.7 filed on Apr. 26, 2021, the contents of which are incorporated herein.

### TECHNICAL FIELD

The disclosure relates to a medical imaging apparatus, in particular a stereo endoscope or a stereo exoscope, comprising a first light source with a first light spectrum and a light source with a second light spectrum, a first optical path with a first optical unit and a first image sensor having a first sensor filter, and a second optical path with a second optical unit and a second image sensor having a second sensor filter, wherein the respective optical path ex-tends between an observation region and the respective image sensor, and the first optical path and the second optical path are arranged spatially offset from one another such that the first image sensor records a first image of the observation region by means of the first optical path and the second image sensor records a second image of the observation region by means of the second optical path, and the first image and the second image are assigned to one another in an overlaid image for the purpose of forming a piece of dual image information, wherein the respective light source is configured to illuminate the observation region with the respective light spectrum such that physiological parameters of the observation region are determinable on the basis of the respective light spectrum.

### BACKGROUND

In this context, endoscopes with two image recording paths in particular, for example stereo endoscopes or stereo exoscopes, which can record a spatial image of an observation region on account of a stereoscopic observation with a first optical path and a second path, are known as medical imaging apparatuses.

Furthermore, endoscopes or else endoscope systems which are able to record and dis-play physiological parameters of an observation region by means of a special illumination with specific light spectra and a corresponding evaluation by means of one or more image sensors are known. For example, so-called multispectral endoscopes are known, by means of which conclusions can be drawn, for example, with regard to an oxygen saturation or else a fat con-tent, a hemoglobin content or any other parameter within the observation region, by way of illuminating the observation region using defined light spectra and correspondingly evaluating the correspondingly radiated-back light spectra. For example, the oxygen content of treated tissue can be directly determined and monitored during a surgical procedure by the use of such a multispectral imaging endoscope.

### SUMMARY

It is an object of the disclosure to improve the prior art.

This object is achieved by a medical imaging apparatus, in particular a stereo endo-scope or a stereo exoscope, comprising a first light source with a first light spectrum and a second light source with a second light spectrum, a first optical path and a first optical unit and a first image sensor having a first sensor filter, and a second optical path with a second optical unit and a second image sensor having a second sensor filter, wherein the respective optical path extends between an observation region and the respective image sensor, and the first optical path and the second optical path are arranged spatially offset from one another such that the first image sensor records a first image of the observation region by means of the first optical path and the second image sensor records a second image of the observation region by means of the second optical path, and the first image and the second image are as-signed to one another in an overlaid image for the purpose of forming a piece of dual image information, wherein the respective light source is configured to illuminate the observation region with the respective light spectrum such that physiological parameters of the observation region are determinable on the basis of the respective light spectrum, wherein the first optical path comprises a first filter with a first filter spectrum and/or the second optical path comprises a second filter with a second filter spectrum, with the result that, from a piece of filtered image information in the first image and/or a piece of filtered image information in the second image, different spectral regions of the respective image are evaluable in order to obtain a piece of additional image information or pieces of additional image information in relation to the physiological parameters in the observation region.

By way of a few components and a simple structure, a medical imaging apparatus de-signed thus combines a dual image endoscope or else a stereoscopic endoscope or a stereoscopic exoscope or any other dual or stereoscopic medical imaging apparatus with the capability of evaluating the observation region with regard to physiological parameters on the basis of the respective light spectrum. In this case, in particular, it is possible, depending on whether a first filter is used in combination with a second filter or a first filter is used on its own or a second filter is used on its own, to perform a spectral evaluation by means of a first filter and/or a second filter and on the basis of a piece of difference information between the first optical path and the second optical path, with the result that the determination of physiological parameters in the observation region is rendered possible using the available imaging technology and without further components, in addition to the dual image.

In this case, forming such a piece of difference information may be necessary, inter alia, since pieces of spectral information correspondingly required for the ascertainment of sampling points are filtered out when a respective filter is used. If different first filters and second filters are used, then the respective missing spectral component can be determined from the respective other optical path, and an alignment can be implemented.

The following terms are explained in this context:

A “medical imaging apparatus” can be any technical and/or electronic device suitable for recording, further processing, and/or transmitting an image of an observation region in medical surroundings, and for example for displaying said image on a visual display unit. By way of example, such a medical imaging apparatus is a dual endoscope, a stereo endoscope, or stereo exoscope. In this case, such a “stereo endoscope” is an imaging apparatus with usually a narrow and elongate design, which is suitable for insertion into a cavity or through a usually small opening and suitable for recording, by means of two cameras or two image sensors, an image of an observation region located within the cavity and/or behind the small opening. A “stereo exoscope” is a comparable device, which is used for example from the outside for imaging purposes during medical procedures, which is to say within the scope of what is known as an open surgical procedure. In this case, the “stereo” property of the respective endoscope or exoscope describes the capability of recording a stereoscopic image of the observation region by means of two optical paths and/or two optical units. A corresponding dual endoscope or dual exoscope is able to record two separate images, without for example a stereoscopic reconstruction being implemented. Attention is drawn in this context to the fact that a respective “endoscope” in the strict sense, as described above, may also be linked within an endo-scope system to further devices, for example a cable guide, further sensors, and/or display equipment for displaying a piece of image information on an external monitor. Further, there frequently is no strict separation between the use of “endoscope” and “endoscope systems”, and these terms are sometimes used synonymously.

A “light source” is for example an LED, an incandescent lamp, or any other light-emitting device. Further, such a light source may also be realized by virtue of a light created by means of an LED or any other light-creating device being steered or directed to a corresponding location at the observation region by means of, for example, a light guide, which is to say for example an optical fiber or an optical fiber bundle. In this case, such a light source serves to illuminate the observation region with light with appropriate light spectra. It is also possible to design the light source as a laser, which is to say with a very narrow light spectrum of only one wavelength in particular, with “one” wavelength in this case denoting a tight region of for example only +/−2 nm or else +/−5 nm, which is to say a range which is significantly narrower than what is obtainable by an LED.

In this case, a “light spectrum” describes the wavelength range and/or an intensity distribution over various wavelengths, in which the respective light source emits light. For example, such a light spectrum can in this case be depicted graphically in the form of a diagram of the illumination intensity against a respective wavelength.

An “optical path” is in particular the path traversed by light of a corresponding image, from the observation region via a respective optical unit to for example a respective image sensor. For example, such an optical path is defined here by an optical axis or as a geometric pro-file.

An “optical unit” denotes the totality of all components which steer light and/or a piece of image information or an image along the optical path. By way of example, such an optical unit comprises lenses, covers, protective screens, or else filters.

By way of example, an “image sensor” is an electronic chip or any other equivalent device, by means of which light running along the optical path and through the respective optical unit and/or a corresponding image can be recorded and converted into electronic signals. By way of example, such an image sensor is a CCD chip or comparable electronic component.

A “sensor filter” describes a filter usually assigned to a respective image sensor or a corresponding filter device, which is suitable for advance filtering of light that is incident on the image sensor and recorded by the image sensor. By way of example, the image sensor comprises a sensor filter which supplies parts of the image sensor assigned to corresponding color values with respective light that has been prefiltered in accordance with the color. Thus, a typical image sensor may for example have an RGB filter in front of corresponding sensor parts for individual pixels for example, with the result that only the respective piece of information in relation to R (red), G (green), and B (blue) is fed to a respective pixel. In this case, every so-called pixel of the image sensor has at least three component pixels, which are each fed the piece of R-, G-, B-information by way of an appropriate filter, with the result that a differentiated color display is made possible by the pixel formed from the component pixels. Inter alia, image sensors with what is known as a Bayer filter are also known in this context.

An “observation region” describes the region, the volume or the area which is intended to be observed by means of the medical imaging apparatus and of which a corresponding image is intended to be taken. By way of example, in this case such an observation region is an organ, a bone, a portion of a human or animal body, or any further region of interest for a corresponding observation.

In this case, “spatially offset from one another” describes two optical paths arranged next to one another in the simplest case, with the result that a respective image can be record-ed from two perspectives. Further, a parallel or skew arrangement of corresponding optical axes along the optical paths can in particular also be configured and used, with the result that it is possible to record what is known as a stereoscopic image. This is made available in an embodiment of the disclosure described hereinafter. In this case, such a stereoscopic image imitates the spatial view of a living being, by virtue of a respective image being recorded from at least two perspectives. Subsequently, a spatial impression can be reconstructed or created from the various pieces of information of the respective images from different perspectives. This is also described as “stereoscopically forming a piece of spatial image information”.

In this case, a “piece of dual image information” is a piece of image information which is put together from two pieces of image information and which for example comprises an image representation of two images recorded of the observation region by means of different optical paths, without these images needing to be stereoscopic.

In this case, a “piece of spatial image information” is the piece of information which allows a conclusion to be drawn, for example with regard to the topography of the observation region and/or a spatial arrangement of corresponding objects within the observation region. In this case, a piece of spatial image information can for example be an image displayed as a 3-D image, which provides an observer with a detailed piece of information about the topography of the observation region. Furthermore, such a piece of spatial information may also arise from the fact that, for example, the respective piece of image information for a respective eye is supplied to an observer, with the result that the actual “spatial formation” is carried out by the observer since spatial vision is physiologically suggested to the observer. By way of example, this can be implemented by means of VR (virtual reality) goggles, which are then supplied with a “piece of dual image information” within the meaning of the description above.

In the present case, “physiological parameters” of the observation region are for example oxygen concentrations, fat contents, perfusion values, hemoglobin concentrations, or else a water content, for example in a considered organ and/or in the tissue of the respective organ in the observation region. By way of example, such physiological parameters are ascertainable by means of corresponding light spectra by virtue of an absorptance for a corresponding wave-length range of the light spectrum being analyzed and this being used to draw conclusions about a corresponding physiological parameter. For example, a certain absorption wavelength is assigned to a hemoglobin concentration, a different absorption wavelength is assigned to a water content, or a third absorption wavelength is assigned to an oxygen content in the blood.

A “filter” is an optical component and hence can be part of the designated optical unit. In particular, the action of a filter is such that certain wavelength ranges of a complete light spectrum are damped or reduced or passed, or else completely hindered from traveling further along the optical path. In this context, such a filter is a corresponding “filter spectrum”, which, in a manner analogous to a light spectrum, describes the respective intensity of passed or re-tained wavelengths of the filter. In this context, reference is made both to a transmission filter spectrum, which describes the intensity component in the respective light spectrum that is passed, and a degree of retention, which specifies what component is not passed by the filter. For example, so-called graduated filters are known in this context; their filtering effect changes continuously over a filter surface. Furthermore, so-called edge filters are known, which respectively retain or pass separated spectral regions quite selectively.

In this case, a “piece of filtered image information” is a piece of image information which has passed through the respective filter, and hence the wavelength components defined according to the respective filter spectrum have been scrubbed or removed therefrom.

In this context, a “spectral region” is a portion or partial region of a corresponding filter range or light spectrum.

A “piece of additional image information” is for example a pictorial representation or an electronic preparation of this pictorial representation, compiled analogously thereto, which allows an observer to draw conclusions about physiological parameters in the observation region. By way of example, such a piece of additional image information is a false color image or a numerical display of corresponding physiological parameters for corresponding picture elements or pixels. In particular, a certain physiological parameter may therefore also mean a displayed numerical value for a pixel selected by an operator, for example.

In order to be able to ascertain a multiplicity of physiological parameters and/or else be able to ascertain certain physiological parameters reliably by means of different light spectra from different light sources, the medical imaging apparatus comprises a third light source with a third light spectrum, a fourth light source with a fourth light spectrum and/or further light sources with further light spectra, wherein the respective light source is configured such that the latter illuminates the observation region with the respective light spectrum, the latter having been adapted to the physiological parameters in particular.

In an embodiment, the first filter spectrum and the second filter spectrum differ from one another, with the result that the first filter and the second filter have different filter spectra.

This arrangement allows the medical imaging apparatus to have a particularly simple structure. As a result of different first filters and second filters, it is possible to create a synergy of the parameters determined in the first optical path and in the second optical path, with the result that, by means of forming the difference between the respective pieces of image information in particular, it is possible to bring about a stereoscopic and spatial representation, and also a representation and/or analysis of corresponding physiological parameters within an image.

In an embodiment, which moreover can be a simplification of the aforementioned embodiment with a first filter spectrum and differently designed second filter spectrum, the first filter spectrum or the second filter spectrum is designed to be substantially completely trans-missive for the image recordable by the first image sensor or for the image recordable by the second image sensor.

Hence, filter spectra that differ from one another can be realized by virtue of using only one, specifically selected filter and also using a filter with a completely transmissive filter spectrum, for example a glass cover in the simplest case, for the respective other image sensor.

In this case, “substantially completely transmissive” describes the property that the filter is so transmissive that the wavelength ranges relevant to the image sensor are passed in full. By way of example, such a filter is then completely transmissive for a wavelength range between 400 nm and 900 nm, with “substantially” in this case also including deviations for technical reasons, which is to say for example wavelength-dependent differences in the transmissivity of 5% or 10% caused by the utilized material and/or other optical properties in particular.

In order also to be able to use synergistic effects between the respective filter and a corresponding sensor filter in this case, the first filter spectrum and/or the second filter spec-trum is chosen to correspond to the first sensor filter and/or correspond to the second sensor filter, with the result that, by means of a respective sensor-filter-dependent sensitivity in different spectral regions, different spectral regions of the respective image are evaluable to obtain a piece of additional image information or a plurality of pieces of additional image information from the observation region.

Thus, for example, it is possible to utilize a high sensitivity of the respective image sensor, which is dependent on the respectively assigned sensor filter, in order to be able to evaluate, in particularly pronounced, fashion a filter spectrum corresponding thereto. By way of example, a high sensitivity of the respective image sensor for a light with a red hue is used in such a way here that the respective filter with its filter spectrum passes a wavelength precisely corresponding thereto, which is to say has a high transmission in a corresponding spectral region. Naturally, this also applies analogously to other light spectra, be they visible or invisible to the human eye.

In a further embodiment, the first filter and/or the second filter is an edge filter or a bandpass filter here.

An “edge filter” has two or more sharply separated spectral regions in which the edge filter transmits, which is to say is transmissive, or absorbs, which is to say is non-transmissive. In this case, such edge filters are implemented as what are known as high-pass filters, which is to say with a transmission in high wavelength regions, or as low-pass filters, which is to say as edge filters with a transmission in low wavelength regions.

A “bandpass filter” is a corresponding filter which comprises a plurality of edge filters, which is to say for example transmits what is known as a “band” of spectral components between a lower wavelength and an upper wavelength. Hence, a separation is implemented with a respective edge filter at each of the lower wavelength and upper wavelength.

In an embodiment, at least one of the respective light spectra has a wavelength of 400 nm to 940 nm, 400 nm to 700 nm, 790 nm to 850 nm, 400 nm to 500 nm, and/or 740 nm to 780 nm. Specifically, an illumination at 940 nm, an illumination with white light from 400 nm to 700 nm, an illumination at 460 nm or an illumination at 770 nm, for example, would be possible here. What should be observed in this context for the present embodiments overall is that a respective wavelength always describes a reference wavelength, which is to say a “nominal wavelength”, and for example also includes a range of decreasing intensities of respectively 10 nm or else 20 nm in an increasing direction and also in a decreasing direction. In particular, this is relevant to the illumination by LEDs or comparable light sources since, as a rule, LEDs are unable to emit light of only one, sharply delimited wavelength and instead create a spectrum with a distributed intensity distribution that drops around a nominal wave-length. It is also noted in this respect that the inventive concept can for example also be implemented using sharply delimited laser radiation from a laser at a certain wavelength, wherein such a laser can implement a significantly narrower range of for example +/−2 nm or +/−5 nm about a nominal wavelength. Corresponding filters can then be embodied with a narrower filter range, which in particular minimizes an influence on a white-light image for an observer.

In any case, different fluorescent substances, tissue reactions, or absorption behaviors of the tissue can be exploited in a targeted manner using such respective light wavelengths.

To be able to also use the medical imaging apparatus for imaging in the visible range and, for example, in order to be able to also use spectral components of an optically uniformly visible illumination, the medical imaging apparatus comprises an additional light source for illuminating the observation region with white light.

“White light”, which is also referred to as “polychromatic light”, describes light which consists of a mixture of different colors, which is to say a mixture of different spectral com-ponents. Such light is therefore also described as being spectrally broadband. In this case, such white light may for example also be white light within the meaning of daylight, but also any other mixture of light wavelengths. By way of example, such white light may also accordingly include, in superimposed form, wavelengths suitable for determining physiological parameters.

In a further embodiment, the formation of the piece of dual image information com-prises a reconstruction of a correlation between the first image and the second image on the basis of a respective piece of filtered first image information and a piece of filtered second image information, wherein the reconstruction is implemented in particular on the basis of pieces of image information with wavelength spectra passed by the first filter and the second filter, in particular by the illumination with white light.

In this case, a “reconstruction of a correlation” describes associating, between the first image and the second image, a picture element accordingly belonging to picture element in the observation region or a corresponding piece of partial information, whereby for example an offset of the first image from the second image is calculated and used as a correction for the formation of the piece of dual image information.

To be able to use the piece of dual image information additionally for an evaluation in respect of the topography or a piece of spatial information, the formation of the piece of dual image information comprises a stereoscopic formation of a piece of spatial image information for the observation region.

In a further embodiment, one of the light sources or a plurality of the light sources illuminates the observation region with a light spectrum corresponding to an adjuvant, with the result that an optical excitation of an adjuvant, in particular a fluorescent substance, is made possible with the aid of the corresponding light spectrum, wherein in particular the first filter and/or the second filter have or has a filter spectrum which is adapted to a light spectrum emitted by the excited adjuvant, in particular fluorescent substance.

By way of example, such an arrangement can serve to create what is known as a fluorescence image, for example an indocyanine green image (ICG image). In the process, an adjuvant, for example a fluorescent substance for an ICG method, is introduced into the observation region. This adjuvant is then excited by the light spectrum corresponding to the adjuvant and emits a light spectrum correspondingly emitted by the adjuvant. For example, if this adjuvant is administered into the blood of a patient as a fluorescent substance, then a perfusion can be determined post excitation by means of the correspondingly emitted light spectrum of the adjuvant.

By way of example, an “adjuvant” in this case is a medicament which is administered to a patient prior to the examination with an endoscope and which is then accumulated in the examined organs and is available there for the evaluation of specific physiological parameters. An example of such an adjuvant is what is known as a contrast agent, which is known as a medical product in various embodiments.

In this context, a “fluorescent substance” is an adjuvant which reacts to an incident light spectrum, the so-called “excitation spectrum”, with the emission of a modified, characteristic light spectrum. Thus, a “fluorescent substance” absorbs light at specific wavelengths and, in response, emits light at the same or different wavelength. Examples of this include sub-stances that shine in blue under what is known as blacklight. Such substances are known in different forms and with different fluorescent colors in both the medical and non-medical field.

If the first filter and/or the second filter in this case have or has a spectrum which is adapted to the light spectrum emitted by the excited adjuvant, then it is possible to clearly delimit a corresponding “response”, which is to say for example the fluorescent reaction of the adjuvant or fluorescent substance, by means of a corresponding filter, and this can be evaluated after damaging spectral components have been scrubbed. In particular, the filter in this case filters out spectral components of the light source which serve to excite the adjuvant. This allows the fluorescence reaction to be evaluated in a manner that is as undisturbed as possible.

In order to implement an illumination of the observation region specifically for corresponding physiological parameters, an excitation filter is assigned to a light source or to a re-spectate light source, wherein the excitation filter filters light emitted by the light source such that the illumination of the observation region with a light spectrum corresponding to the adjuvant is made possible.

By way of example, such an excitation filter can be designed in such a way as a band-pass filter in this case that only a small spectral band which excites the adjuvant is passed. Consequently, a targeted excitation of the adjuvant, for example the fluorescent substance, is made possible.

In a further embodiment, the medical imaging apparatus comprises an evaluation unit which is configured to evaluate the first image and/or the second image in relation to an OHI, TWI, StO2 and/or NIR index, with the result that a hemoglobin content, a water content, an oxygen concentration and/or a presence of an adjuvant, in particular a fluorescent substance, is determinable.

By way of example, such an “evaluation unit” comprises a memory in this case, in which a method for evaluating the medical imaging apparatus is stored. Furthermore, the evaluation unit may comprise a processor or a microcontroller, wherein the evaluation method is executable using the processor or the microcontroller.

In order to present for example both the piece of stereoscopic image information and the pieces of further image information to a corresponding user in a convenient and evaluable fashion, the medical imaging apparatus comprises a display unit which is configured for simultaneous, superimposed and/or correlated display of the piece of first image information, the piece of second image information, the stereoscopically formed piece of spatial image information and/or the piece of additional image information.

In this case, such a “display unit” is for example a PC, a minicomputer or a visual dis-play unit with a corresponding processor, which is able to display both the piece of first image information, the piece of second image information, a piece of spatial image information formed therefrom, and/or also a piece of additional image information in the form of for ex-ample a hemoglobin content of the observation region. For example, such hemoglobin content is superimposed on the other pieces of image information as a color image pixel-by-pixel in this case. In this case, the display unit may also comprise the evaluation unit therein, with the result that there is no need for separate equipment for the display unit and the evaluation unit.

The disclosure is explained in more detail using exemplary embodiments.

### BRIEF DESCRIPTION OF DRAWINGS

FIG. 1 shows a schematic illustration of a tip of a medical stereo endoscope,

FIG. 2 shows a schematic illustration of the tip of the medical stereo endoscope of FIG. 1 in a cut side view,

FIG. 3 shows a diagram regarding different absorption rates of physiological parameters in an exemplary tissue,

FIG. 4 shows a diagram regarding different color sensitivities of an image recording chip,

FIG. 5 shows a diagram of a filter spectrum of an exemplary filter for the tip of the stereo endoscope of FIGS. 1 and 2, and

FIG. 6 shows a diagram regarding the time sequence of illumination schemes for the stereo endoscope.

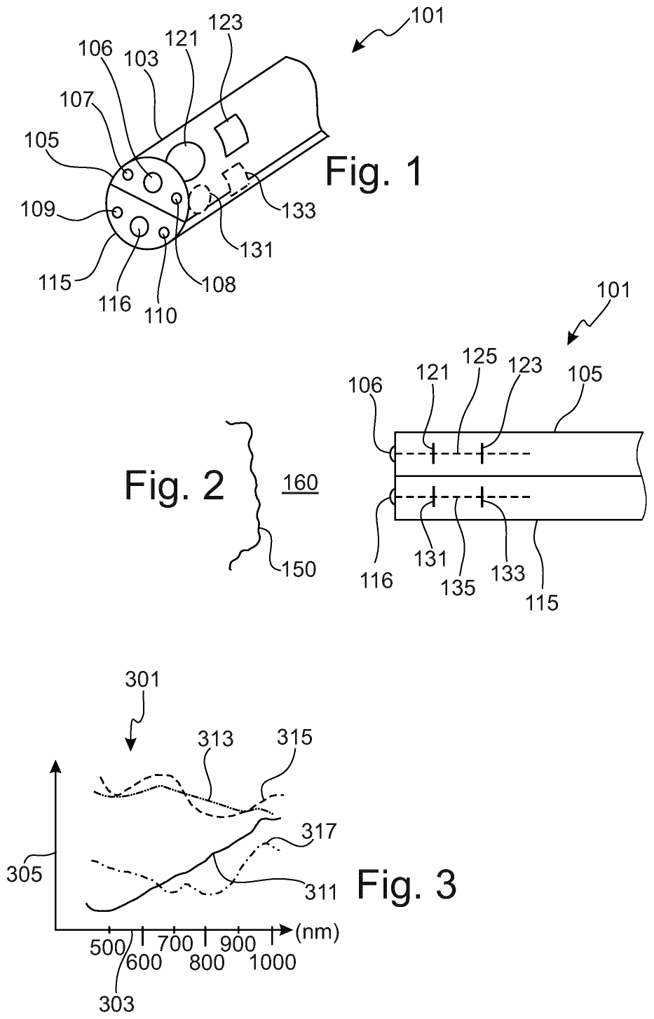

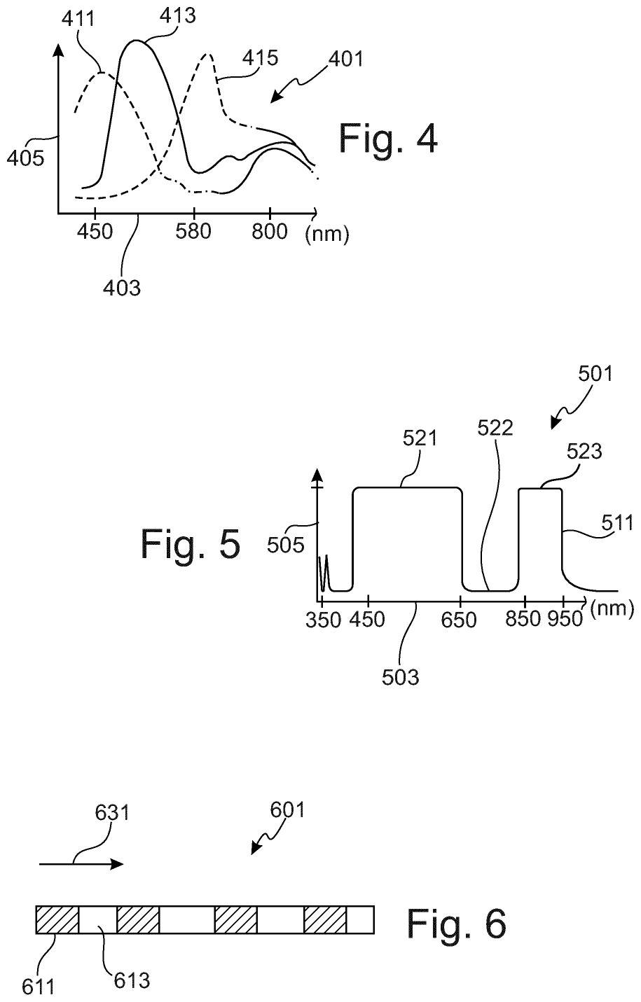

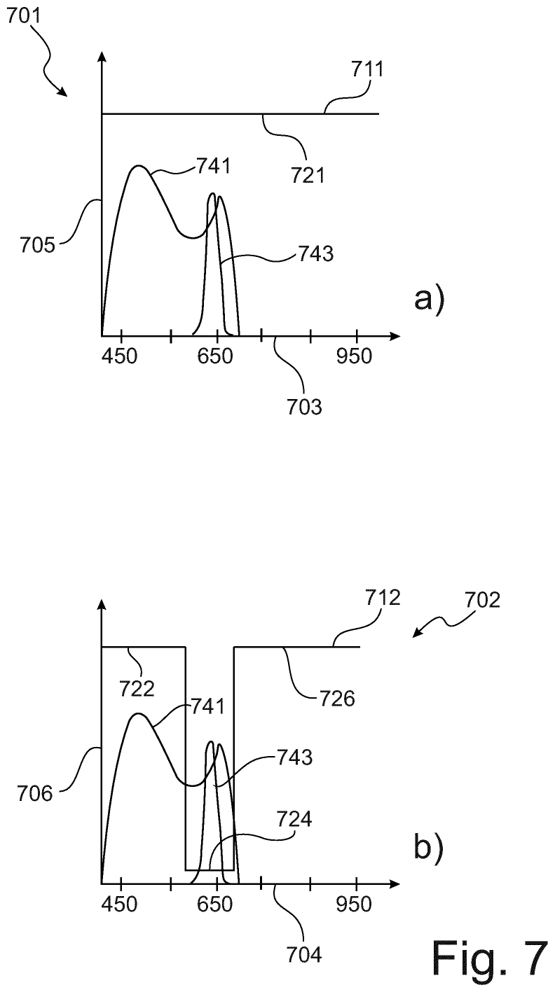

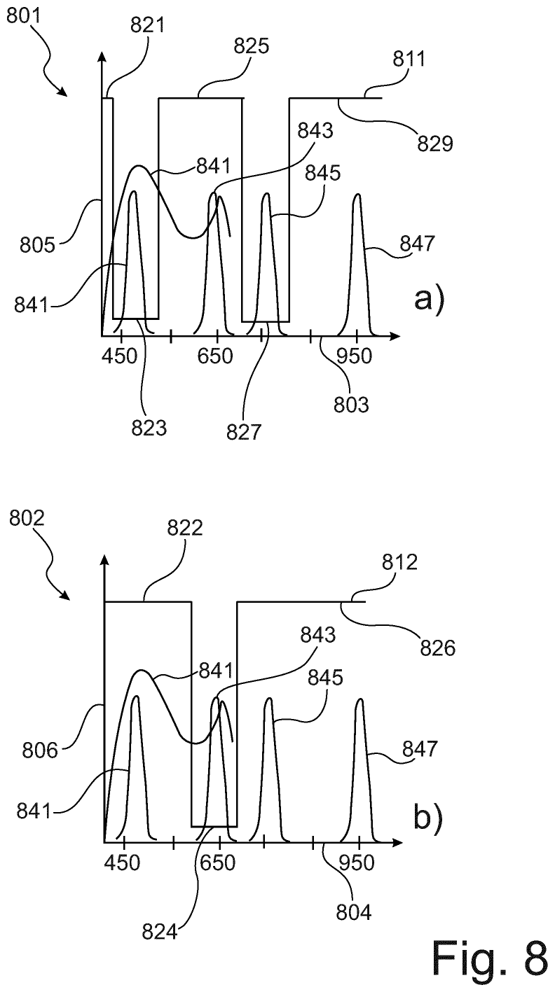

### DETAILED DESCRIPTION OF PREFERRED EMBODIMENTS

A stereo endoscope 101 comprises a tip 103. In this case, the tip 103 is formed from a first part 105 and a second part 115. In this case, the first part 105 accommodates all components for a first optical path of a stereoscopic observation; the second part 115 accommodates all components for a second optical path for a stereoscopic observation.

To this end, the first part 105 comprises a lens 106, which guides an incident piece of image information onto an image sensor 123 through a filter 121. During a surgical procedure, the image of an exemplary organ 150 located in an observation region 160 is imaged on the image sensor 123 by means of this optical unit formed by the lens 116 and the filter 121.

In the second part 115, a second image of the organ 150 in the observation region 160 is imaged on an image sensor 133 in an analogous manner by means of a lens 116 and a filter 131. In this case, the lens 106, the filter 121, and the image sensor 123 are located on one optical axis 125; the lens 116, the filter 131, and the image sensor 133 are located along an optical axis 135. In this case, the lens 106 and the lens 116 view the organ 150 from a position parallel to one another, with the result that a stereoscopic image of the organ 150 in the observation region 160 is able to be created in a corresponding evaluation unit (not depicted here) or in VR goggles (not depicted here) by means of the first part 105 and the second part 115. To this end, the piece of image information created on the image sensor 123 and the piece of image information created on the image sensor 133 are transmitted to the evaluation unit (not depicted here) and reconstructed to form a dual image, wherein the offset between the two images is removed by calculation in order to be able to relate respective pieces of information from the images to a corresponding point on the organ 150. In this context, reference is also made to the reconstruction of the disparity. As a rule, this is implemented on the basis of a white-light image and on the basis of a respective calibration of the image sensor 123 and image sensor 133.

LEDs 107 and 108 are arranged on the first part 105 and serve the purpose of illuminating the organ 150 in the observation region 160. Additionally, LEDs 109 and 110 are arranged on the second part 115. In this case, the LEDs 107, 108, 109 and 110 are only arranged geometrically on the first part 105 and on the second part 115, and initially specify no specific assignment to the respective optical axes 125 and 135. In this case, the LEDs 107, 108, 109 and 110 are selected and configured in the specified order as narrowband LEDs for illuminating the organ 150 in the observation region 160 using wavelengths of 450 nm, 580 nm, 700 nm, and 800 nm. In this case, a full width at half maximum of less than 20 nm, for example less than 10 nm, is referred to as narrowband, wherein this may vary depending on application and the desired precision of corresponding recordings. By way of example, further LEDs for illuminating the organ 150 may also emit wavelengths of 460 nm, 660 nm, 765 nm or else 940 nm (these LEDs are not depicted here). In this case, too, specifications regarding wavelengths with the above-described full widths at half maximum and/or deviations with respect to a specifically given wavelength are taken as read, for example such that an adaptation of the specified 765 nm to 770 nm is possible, for example if this makes it possible to obtain a clearer or more precise reaction of an illuminated tissue.

The filter 121 and the filter 131 have different filter spectra from one another. In an alternative, it is also possible for only the filter 121 or only the filter 131 to be present, with the respective other optical axis then having no filter. In this example, the filter 131 is completely transmissive, that is to say optically without an influence.

A diagram 301 has a wavelength axis 303 and an intensity axis 305. In this case, the diagram 301 describes the absorptance of different tissue constituents of the object 150 with respect to the respective light wavelength. To this end, the intensity is plotted on an intensity axis 305 against the wavelength on the wavelength axis 303. Herein, an absorption curve 311 shows the absorption of water, an absorption curve 313 shows the absorption of deoxygenated blood, the absorption curve 315 shows the absorption of oxygenated blood, and the ab-sorption curve 317 shows the absorption of fatty tissue, in each case in relation to the respective light wavelength on the wavelength axis 303.

A diagram 401 has a wavelength axis 403 and an intensity axis 405. The diagram 401 describes what is known as an RGB sensitivity of an image sensor, which is to say for example of the image sensor 123 and/or image sensor 133. In this case, the image sensor 123 and the image sensor 133 are configured as image sensors with a Bayer filter, with the result that different component pixels are able to record different pieces of color information for each pixel with the sensitivity curves for different colors shown in the diagram 401. In this context, reference is also made below to “channels”, which is to say a “red channel”, a “green channel”, and a “blue channel”, with the sensitivity centroid for R (red), G (green) and B (blue) for the respective component pixel being denoted in each case. In this case and in a manner analogous to diagram 301, an intensity on the intensity axis 405 is plotted against a wavelength on the wavelength axis 403. In this case, a sensitivity curve 411 describes the sensitivity of an image sensor in the blue channel, a sensitivity curve 413 describes said sensitivity in the yellow channel, and a sensitivity curve 415 describes said sensitivity in the red channel, for incident light. It is qualitatively recognizable here that a respective sensitivity curve has a local maximum in respect of a corresponding wavelength range, which is to say it is particularly sensitive for the evaluation in respect of a respective color. However, it is recognizable that, for example, other pieces of information to the pieces of yellow light information are also recorded in the sensitivity curve 413, and are converted into a corresponding, weaker signal. The same also applies analogously to the other sensitivity curves relating to other colors.

A diagram 501 has a wavelength axis 503 and a transmission axis 505. In this case, the diagram 501 describes the transmission, which is to say the transmissivity, of a corresponding filter, for example of the filter 121 or filter 131, in exemplary fashion. In this case, a transmission curve 511 has a pronounced transmission spectrum 521, a pronounced blocking spectrum 522, and a pronounced further transmission spectrum 523. Hence, the filter on which the diagram 501 is based is a dual bandpass filter. In this case, the wavelength range between approx. 450 nm and approx. 650 nm, and between approx. 820 nm and approx. 950 nm is passed; other wavelength ranges are blocked virtually in full. In this case, “complete” describes a significant, technically advantageous attenuation of the transmitted light with a factor of for example more than 100 vis-a-vis a pass, or else for example a factor of more than 1000.

A block diagram 601 shows the time sequence of an illumination sequence for the stereo endoscope 101. In this case, white-light phases 611 and MSI phases 613 are driven alternately. The organ 150 is illuminated by white light by means of a further LED (not depicted here) in the white-light phase and illuminated by the above-described light wavelengths for the multi-spectral imaging (MSI) by means of the LEDs 105, 108, 109 and 110 in the MSI phase 613. In this case, the different phases are driven alternately along the time axis 631, to be precise with a respective exposure time of approx. 25 ms for the MSI and approx. 8 ms for the white light in the present example. In the process, there is a synchronized evaluation of the image sensor 123 and image sensor 133, with the result that it is possible to assign the signals of the respective image sensor to the respective phase along the time axis 631. The disparity of the two different images is also reconstructed in an MSI phase 613, as described above.

The use of the stereo endoscope 101 for determining certain physiological parameters of the organ 150 and, for the observer, simultaneously determining ICG parameters is intend-ed to now be described below.

In this case, the filter 121 is assumed to be arranged along the optical axis 125, with the filter 121 being an ICG filter, which is to say a filter for determining an indocyanine green fluorescence of a corresponding adjuvant in the organ 150. The filter 121 behaves according to the transmission curve 511 of the diagram 501, which is to say it largely passes light wave-lengths between approx. 400 nm and approx. 650 nm, and light wavelengths between approx. 820 nm and approx. 950 nm. Other wavelengths are virtually completely retained and do not in the process penetrate significantly to the image sensor 123. In this example, the filter 131 is designed as a fully transparent filter 131 along the optical axis 135, which is to say said filter is not optically relevant, with the result that pieces of unfiltered image information relating to the organ 150 are incident on the image sensor 133.

An ICG value and physiological parameters of the organ 150 are then determined directly successively in successive frames of the image recording by means of the image sensor 123 and by means of the image sensor 133. In this case, illuminations for the ICG image and for the MSI image, as are required alternately, are ensured by means of the switchover of the illumination by means of the LEDs. The switchover is implemented in accordance with the block diagram 601 and over such a short period of time that it is not perceived by an observer. As a result, no movement artifacts arise due to the switchover of the illumination and the al-ternate readout of the image sensors 123 and 133, even if the endoscope is moved in the observation space 160. Said artifacts would arise during the stereo reconstruction of the two created images with respect to one another in particular.

In the context of the sensitivity of the respective image sensor with respect to different wavelengths, the following pieces of information, for example, can be read out and/or calculated virtually simultaneously from the different illuminations according to the block diagram 601 and the difference between the filter 121 and the free path for the light along the path where the filter 131 is completely transmissive. This is implemented during a respective MSI phase of the illumination and is explained in this context on the basis of a parallel determination of a hemoglobin index (OHI) and an oxygen saturation index (StO2).

To this end, an evaluation of the red channel of the image sensor 123 supplies a measured intensity at 580 nm, since the sensitivity of the respective red-sensitive component pixel of the respective image sensor is greatest for the light wavelength of 580 nm here. Further-more, the light at 580 nm reaches both the image sensor 123 and the image sensor 133 since the filter 121 is also transmissive to this light wavelength according to diagram 301. The intensity for 580 nm, 700 nm, and also 800 nm is determined on the respective red component pixel in the image sensor 133.

An evaluation of the green channel of the image sensor 123 supplies a measured intensity at 450 nm and at 580 nm. Furthermore, the light at 450 nm and 580 nm reaches both the image sensor 123 and the image sensor 133 since the filter 121 is also transmissive to those light wavelengths according to diagram 501. Light at a wavelength of 700 nm is blocked by the filter 121, but light at a wavelength of 800 nm would in turn be passed (cf. diagram 501), but is not read out in this case. The intensity for 450 nm, 580 nm, 700 nm, and also 800 nm is determined on the respective green component pixel in the image sensor 133. Attention is drawn to the fact that a corresponding wavelength in this case naturally also includes a certain full width at half maximum of for example 15 nm; the “wavelength” should be understood here as a nominal wavelength. However, for the understanding of the disclosure, reference is made here to a “wavelength” with an assumed clear selectivity, which in technological reality comprises a certain deviation.

To this end, an evaluation of the blue channel of the image sensor 123 supplies a measured intensity at 450 nm. The light at 450 nm reaches the image sensor 123 since the filter 121 is also transmissive to this light wavelength according to diagram 301. The intensity for 450 nm and also 800 nm is determined on the respective blue component pixel in the image sensor 133.

In summary, the following intensities are available, determined using the image sensor 123: the intensity for 580 nm using the red channel, for 450 nm and 580 nm using the green channel, and for 450 nm using the blue channel.

In summary, the following intensities are available, determined using the image sensor 133: the intensity for 580 nm, for 700 nm and for 800 nm using the red channel, for 450 nm, for 580 nm and for 800 nm using the green channel, and for 450 nm and for 800 nm using the blue channel.

Now, to determine the physiological parameters, OHI and StO2 in this example, the various pieces of information are combined with one another and hence rendered usable:

At a sampling point at 450 nm, the blue channel of the sensor 123 is used to check the parameters as determined below: This light wavelength is located at what is known as the isobestic point for both the OHI value (hemoglobin) and for the StO2 value (oxygen saturation). This isobestic point is characteristic for the measured intensity at this wavelength not depending on, or only depending very slightly on, the oxygen content in the observation region. This can be used to distinguish between changes in the OHI value and in the StO2 value.

At a sampling point of 580 nm, the OHI parameter is determined by means of the red channel of the sensor 123. At this light wavelength, both the sensitivity curve 415 and the absorption curve of the respective hemoglobin parameter are at a maximum, with the result that it is possible to determine the OHI value reliably and with good selectivity with respect to other wavelength ranges.

The StO2 value is determined at a sampling point with a light wavelength of 700 nm by way of a calculation. It should be observed in this context that the image sensor 123 receives no pieces of image information regarding the light wavelength of 700 nm on account of the filter 121 with the transmission curve 511. Therefore, the underlying light intensity of the respective channels of the respective RGB image sensor is calculated as follows:

P(R2)−R1−P(B2)+B1
where the following applies:
R1: intensity of the red channel of the image sensor 123
R2: intensity of the red channel of the image sensor 133
B1: intensity of the blue channel of the image sensor 123
B2: intensity of the blue channel of the image sensor 133
P: projection rule (correction factor for the disparity)

In this case, the projection rule P is derived from the reconstruction of the disparity, as described above. In this case, the factor P describes intensity differences between the image of the image sensor 123 and the image of the image sensor 133 for respective pixels.

Furthermore, an StO2 value and an OHI value can be determined by means of the dependence:

P(B2)−B1

In the present case, such sampling points are wavelength ranges from the absorption curves 311, 313, 315 and 317 shown in the diagram 301, selected in such a way that a statement about for example the hemoglobin content in the organ 123 is made possible from respectively one, two or more sampling points.

The ICG parameters, which is to say the fluorescence response of the organ 150, are determined by means of the image sensor 123 in the respective ICG phases according to block diagram 601. To this end, there is an excitement at a light wavelength of 700 nm, with fluorescence arising at a light wavelength of approx. 600 nm. In this case, the filter spectrum of the filter 121 is chosen in accordance with the transmission curve 511 such that the bother-some influence of the light of the LED 109 at 700 nm is filtered out but the light of the fluorescence at approx. 600 nm is incident virtually without impediment on the image sensor 123, with the result that the fluorescence for carrying out the ICG examination can be evaluated as exactly as possible.

Overall, with direct temporal proximity of corresponding frames during the image re-cording by means of the image sensor 123 and image sensor 133, this renders possible a virtually parallel determination of both the physiological parameters by means of MSI and the parameters of an ICG examination, with movement artifacts being prevented to the best possible extent.

For the following, supplementary examples, the image sensor 123 is referred to as “left” channel and the image sensor 133 is referred to as “right” channel.

A diagram 701 has a wavelength axis 703 and an intensity axis 705. In this case, the diagram 701 describes, in exemplary fashion, the transmission, which is to say the transmissivity, of a corresponding filter, the filter behavior being described by a transmission curve 711. The transmission curve 711 represents a transmission spectrum 721 of the corresponding filter, to be precise in the case of the diagram 701 for the left channel of a stereo endoscope.

Moreover, the diagram 701 has an illumination curve 741 for representing the illumination of the observation region 160 using white light and an illumination curve 743 for representing the illumination of the observation region 160 using visible red light as excitation light. In this case, the transmission spectrum 721 ensures that all wavelength ranges along the full spectrum represented along the wavelength axis 703 from approximately 400 nm to approximately 1000 nm are passed and are recordable by the corresponding image sensor.

Analogously, a diagram 702 with a wavelength axis 704 and an intensity axis 706 shows the conditions for the right channel, specifically a transmission curve 712 which exhibits a transmission spectrum 722 from approximately 400 nm to approximately 640 nm, a blocking spectrum 724 from approximately 640 nm to approximately 680 nm, and adjoining the latter a transmission spectrum 726 from approximately 680 nm to approximately 1000 nm. In the context of the illumination curve 741 and the illumination curve 743, which are also depicted in the diagram 702, it is therefore evident that the substantial component of the visible red-light illumination and a corresponding red region of the white-light illumination in particular are filtered out. These wavelength regions are therefore not identifiable by a corresponding right channel.

Imaging with a corresponding visible red light is then carried out in a manner analogous to the block diagram 601, but with alternate illumination in successive frames of the imaging using white light and visible red light at a wavelength of 660 nm for a red-light fluorescence. In this case, an optical response of the organ 150 to the excitation light at 660 nm is implemented at a wavelength of approximately 700 nm and also at longer wavelengths. In a first frame, in which illumination is provided by means of white light, which is to say light at a wavelength of between 400 nm and 700 nm, a complete white-light image not influenced by a filter becomes visible in the left channel, specifically in a manner analogous to the transmission spectrum 721. In the right channel, by contrast, the red component between 640 nm and 680 nm of the white light is filtered out by the blocking spectrum 724. Using corresponding green components and also blue components of a corresponding image from the right channel and the completely present image of the left channel, it is then possible to reconstruct the image by correlating corresponding pixels. In this case, this correlation is implemented using known methods, for example a pixel comparison, a comparative search for geometric arrangements in the image, or the like. As a result, the disparity between different images can be re-moved by calculation. Hence, what is known as a stereo reconstruction is carried out. A corresponding wavelength range between 640 nm and 680 nm, which is to say in particular the red component of the image without discrete resolution of individual wavelengths, from the left channel in a manner analogous to diagram 701 can then be converted for the right channel by way of this stereo reconstruction and then complements corresponding image components computationally, with the result that a corresponding white-light image is visible within the corresponding frame in both channels, which is to say for example a complete stereo image with white-light illumination can be displayed for an operator.

Then, there is an illumination using visible red light at a wavelength of 660 nm for a red-light fluorescence in a second frame, which follows the just-described frame.

Then, a corresponding red-light fluorescence component at approximately 700 nm is so weak in the left image that it is swamped by corresponding light components, or it is not perceivable on account of a signal-to-noise ratio and a correspondingly weak fluorescence signal.

In the right channel, by contrast, a corresponding red-light fluorescence is visible as a result of the clear filtering-out of the red-light excitation light at 660 nm, wherein the fluorescence signal can now be converted from the right channel to the left channel by way of the stereo reconstruction described above, and hence a display of the fluorescence signal for an operator is now possible in both channels. As a result of a correspondingly quick succession of frames and the respective alternate illumination with white light and visible red light, the respective images can consequently be displayed stereoscopically for an operator in each case.

In analogous fashion, this configuration example can be carried out using what is known as fluorescein fluorescence rather than red-light fluorescence, with corresponding filters being matched to the corresponding excitation light. In this case, the red channel can then be used for pixel correlation and stereo reconstruction, with a green channel and a blue channel being alternately replaced by calculation.

A further example is described below:

A diagram 801 with a wavelength axis 803 and an intensity axis 805 shows a transmission curve 811 for the left observation channel. The transmission curve 811 has a transmission spectrum 821 between 400 nm and 440 nm, which is adjoined by a blocking spectrum 823 from 440 nm to 480 nm. Subsequently, the transmission curve 811 in turn has a transmission spectrum 825 between 480 nm and 750 nm and an adjacent blocking spectrum 827 filters out light between 750 nm and 790 nm, with a transmission spectrum 829 once again transmitting light above 790 nm. Furthermore, an illumination curve 841 for white light, an illumination curve 843 for visible red light, an illumination curve 845 for indocyanine green excitation light, an illumination curve 847 for multi-spectral illumination, and an illumination curve 849 for fluorescein illumination are depicted in a manner analogous to diagram 701 and 702.

In a manner analogous thereto, these respective illumination curves are also plotted in a diagram 802 for the right channel, with the diagram 802 having a wavelength axis 804 and an intensity axis 806. A transmission curve 812 represents the filter properties of a corresponding filter for the right channel, specifically a transmission spectrum 822 between 400 nm and 640 nm, a blocking spectrum 824 between 640 nm and 680 nm, adapted to the excitation light in the visible red range, and a transmission spectrum 826 between 680 nm and 1000 nm.

In a manner analogous to the example explained on the basis of diagrams 701 and 702, the illumination can also alternate here between white light and visible red light for a red-light fluorescence, with a corresponding conversion of the filtered-out amount of light in the re-spectate channels being implemented in analogous fashion. Hence, red-light fluorescence in combination with white-light imaging in a stereoscopic display is also possible in the case of the filter arrangement according to diagrams 801 and 802.

Likewise, illumination can alternately be implemented with white light and with excitation light for indocyanine green fluorescence, with this excitation light then being formed at 765 nm or else for example 770 nm wavelength, depending on the indocyanine green fluorescent substance used. Once again, there is illumination with white light and with indocyanine green fluorescence excitation light in different, successive frames, with white light without a blue component appearing in the left image and white light without a red component appearing in the right image during the white-light illumination. Then, a stereo reconstruction analogous to the above embodiment can be described by means of the green components of the left and the right image. The red component of the left image can then be transferred computationally to the right image and the blue component of the right image can be transferred computationally to the left image, with the result that a complete, stereoscopic white-light image arises.

An indocyanine green fluorescence between 790 and 850 nm can be perceived in the left image during the time in which there is an illumination with indocyanine green excitation light at 765 nm or 770 nm. Then, the excitation light is recorded in full in the right image, as well as a comparatively very small component of indocyanine green fluorescence which is thus lost in the noise in the right image. Thereupon, the indocyanine green component of the fluorescence signal can be transferred from the left to the right channel, to be precise on the basis of the stereo reconstruction implemented in the white-light frame.

In a manner analogous thereto, there can be a virtually simultaneous display of a white-light image and a fluorescein fluorescence image, by virtue of white-light illumination being implemented in the first frame, the left image being recorded without a blue component and the right image being recorded without a red component, and a corresponding correlation, stereo reconstruction, and transfer of corresponding color components being implemented in a manner analogous to the previous example.

In a subsequent frame, a fluorescein excitation at 460 nm is carried out, with a corresponding fluorescence at 500 nm being perceivable in the left image. In exchange, the fluorescein excitation light at 460 nm is filtered out of the left image.

The excitation light at 460 nm is visible in the right image; this is displayed by the illumination curve 849. A fluorescence component at 500 nm is accepted in the right channel; however, it is significantly weaker than other signals and therefore lost in the noise. The fluorescein fluorescence signal can then be transferred from the left image to the right image, in a manner analogous to the preceding stereo reconstruction comparison.

A virtually simultaneous operation of white-light imaging and multi-spectral imaging can be implemented in a manner analogous thereto, wherein there is white-light illumination in a manner analogous to the embodiment above in a first frame, with a stereo reconstruction and the transfer of the corresponding color components between the images also being implement-ed here.

In a further frame there then is narrowband illumination for a multi-spectral analysis with wavelengths of 660 nm and 940 nm, with the reflection spectrum of both 660 nm and 940 nm being visible in the left image. Then, spectral separation can be implemented by the recording behavior of the RGB sensors. Furthermore, MSI parameters can be calculated.

The reflection spectrum at 940 nm is perceived in the right channel, wherein the multi-spectral signal can be transferred from the left channel to the right channel by way of a trans-formation from the white-light frame. Hence, a virtually simultaneous display of white-light imaging in a stereoscopic representation and a corresponding tissue analysis is possible in the respective mode, provided corresponding frames are successively displayed above a frequency that is perceivable by a person.

The intention is to also explain a further, combined mode with reference to diagrams 801 and 802.

In this case, the illumination with white light with the corresponding transfers of spectral components is implemented in a first frame, and a stereo reconstruction and corresponding correlation of left to right image on the basis of the green component. Furthermore, the red component and the blue component can be transferred on the basis of the examples above.

Then, in a further frame, an illumination can be implemented at 660 nm as a possible multi-spectral wavelength, and also as an illumination with visible red light for a red-light fluorescence, with the evaluation being implemented in a manner analogous to the procedure presented above.

A further frame then for example uses an illumination wavelength at 765 nm, which can then for example be used for multi-spectral analysis.

The core idea of each of these exemplary embodiments lies in the transfer of corresponding image components, for example for tissue analyses, from one channel to the respective other channel and hence in the enablement of an apparent stereoscopic display of these analysis images, for example by virtue of a stereo reconstruction from a white-light image being used for a computational correlation of corresponding channels. In a manner analogous thereto, the transfer of corresponding color components from respective white-light images of different channels with different transmission behaviors of the respective filters can be used to reconstruct a complete stereoscopic white-light image.

At this point, reference is made to the fact that the LEDs used for illumination purposes can be substituted, either on an individual basis or else in the totality thereof, by respective lasers, for example to implement an illumination with very narrowband light around 460 nm and 660 nm using corresponding lasers rather than LEDs. In a manner analogous to the block-in spectra 823 and 827, use could then be made of very narrowband filters, specifically what are known as notch filters. What is advantageous here is that, at least for the white-light image, the filtered-out spectra of the excitation light are so narrowband that it is even possible to dispense with a reconstruction of the white-light image for these narrow wavelength ranges while an optically appealing white-light image is nevertheless generated by virtue of the missing narrowband wavelength ranges not being identifiable by an observer.

1. A medical imaging apparatus, in particular a stereo endoscope or stereo exoscope, the medical imaging apparatus comprising:
a first light source with a first light spectrum;
a second light source with a second light spectrum;
a first optical path with a first optical unit and a first image sensor having a first sensor filter;
a second optical path with a second optical unit and a second image sensor having a second sensor filter;
wherein a respective first and second optical path extends between an observation region and the respective first and second image sensor, and the first optical path and the second optical path are arranged spatially offset from one another such that the first image sensor records a first image of the observation region by means of the first optical path and the second image sensor records a second image of the observation region by means of the second optical path, and the first image and the second image are assigned to one another in an overlaid image for the purpose of forming a piece of dual image information, wherein the respective light source is configured to illuminate the observation region with the respective light spectrum such that physiological parameters of the observation region are determinable on the basis of the respective light spectrum, characterized in that the first optical path comprises a first filter with a first filter spectrum and/or the second optical path comprises a second filter with a second filter spectrum, with the result that, from a piece of filtered image information in the first image and/or a piece of filtered image information in the second image, different spectral regions of the respective image are evaluable in order to obtain a piece of additional image information or a plurality of pieces of additional image information in relation to the physiological parameters in the observation region.

2. The medical imaging apparatus as claimed in claim 1, further including a third light source with a third light spectrum, a fourth light source with a fourth light spectrum and/or further light sources with further light spectra, wherein the respective light source is configured such that the latter illuminates the observation region with the respective light spectrum, the latter having been adapted to the physiological parameters in particular.

3. The medical imaging apparatus as claimed in claim 1, wherein the first filter spectrum and the second filter spectrum differ from one another, with the result that the first filter and the second filter have different filter spectra.

4. The medical imaging apparatus as claimed in claim 3, wherein the first filter spectrum or the second filter spectrum is designed to be substantially completely transmissive for the image recordable by the first image sensor or for the image recordable by the second image sensor.

5. The medical imaging apparatus as claimed in claim 1, wherein the first filter spectrum and/or the second filter spectrum corresponds to the first sensor filter and/or to the second sensor filter, with the result that, by means of a respective sensor-filter-dependent sensitivity in different spectral regions, different spectral regions of the respective image are evaluable to obtain a piece of additional image information or a plurality of pieces of additional image information from the observation region.

6. The medical imaging apparatus as claimed in claim 1, wherein the first filter and/or the second filter is an edge filter or a bandpass filter.

7. The medical imaging apparatus as claimed in claim 1, wherein at least one of the respective light spectra has a wavelength of 400 nm to 940 nm, 400 nm to 700 nm, 790 nm to 850 nm, 400 nm to 500 nm, and/or 740 nm to 780 nm.

8. The medical imaging apparatus as claimed in claim 1, further including an additional light source for illuminating the observation region with white light.

9. The medical imaging apparatus as claimed in claim 1, wherein the formation of the piece of dual image information comprises a reconstruction of a correlation between the first image and the second image on the basis of a respective piece of filtered image information, wherein the reconstruction is implemented in particular on the basis of pieces of image information with wavelength spectra passed by the first filter and the second filter, in particular by the illumination of the observation region with white light.

10. The medical imaging apparatus as claimed in claim 1, wherein the formation of the piece of dual image information comprises a stereoscopic formation of a piece of spatial image information for the observation region.

11. The medical imaging apparatus as claimed in claim 1, wherein one of the light sources or a plurality of the light sources illuminates the observation region with a light spectrum corresponding to an adjuvant, with the result that an optical excitation of an adjuvant, in particular a fluorescent substance, is made possible with the aid of the corresponding light spectrum, wherein in particular the first filter and/or the second filter have or has a filter spectrum which is adapted to a light spectrum emitted by the excited adjuvant, in particular fluorescent substance.

12. The medical imaging apparatus as claimed in claim 11, further including an excitation filter assigned to at least one of the first light source, the second light source, third light source and fourth light source, wherein the excitation filter filters light emitted by the respective first light source, the second light source, third light source and fourth light source such that the illumination of the observation region (160) with a light spectrum corresponding to the adjuvant is made possible.

13. The medical imaging apparatus as claimed in claim 1, further including an evaluation unit which is configured to evaluate the first image and/or the second image in relation to an OHI, TWI, StO2 and/or NIR index, with the result that a hemoglobin content, a water content, an oxygen concentration and/or a presence of an adjuvant, in particular a fluorescent substance, is determinable.

14. The medical imaging apparatus as claimed in claim 1, further including a display unit which is configured for simultaneous, superimposed and/or correlated display of the piece of first image information, the piece of second image information, the piece of dual image information, the stereoscopically formed piece of spatial image information and/or the piece of additional image information.
